(19) United States
(12) Patent Application Publication
(10) Pub. No.: US 2024/0373869 A1
(43) Pub. Date: Nov. 14, 2024

(54) PROCESS FOR THE PREPARATION OF A FERMENTED MILK PRODUCT

(71) Applicant: SOCIETE DES PRODUITS NESTLE S.A., Vevey (CH)

(72) Inventors: LAURENT FERRIER, Servion (CH); CARINE BLANCHARD, Le Mont-sur-Lausanne (CH); VIRGINIE KAPCHIE, Ada, MI (US); ANA LUCIA VECELLIO, Spring Lake, MI (US); GABRIELA MORAMAY AGUILAR-LOPEZ, Grand Rapids, MI (US)

(21) Appl. No.: 18/692,170

(22) PCT Filed: Sep. 15, 2022
(86) PCT No.: PCT/EP2022/075685

§ 371 Date: Mar. 14, 2024

(30) Foreign Application Priority Data
Sep. 17, 2021 (EP) 21197501.6

(51) Int. Cl. A23C 9/123; A23C 1/04; A23C 9/13; A23C 9/133; A23L 33/00; A61K 35/745; A61K 35/747; C12N 1/20; C12R 1/01; C12R 1/225

(52) U.S. Cl. CPC A23C 9/1234; A23C 1/04; A23C 9/1232; A23C 9/1307; A23C 9/1315; A23C 9/133; A23L 33/40; A61K 35/745; A61K 35/747; C12N 1/20; A23V 2400/175; A23V 2400/531; C12R 2001/01; C12R 2001/225

(57) ABSTRACT

The present invention concerns a process for the preparation of a fermented milk product B. lactis and L. rhamnosus, for example a fermented milk powder, to the fermented milk product itself and to nutritional compositions comprising it. The present invention also relates to the use of such fermented milk products, optionally in a nutritional composition.

### FIELD OF THE INVENTION

The present invention concerns a process for the preparation of a fermented milk product, for example a fermented milk powder, to the fermented milk product itself and to nutritional compositions comprising it. The fermented milk product and nutritional composition comprising it have increased palatability. The present invention also relates to the use of the fermented milk product, optionally in a nutritional composition, to promote nutrients absorption and gut health. The present invention also relates to the use of a nutritional composition comprising a fermented milk powder according to the present invention to promote nutrient absorption, an healthy microbiota and gut health.

### BACKGROUND OF THE INVENTION

Immune and gut health are among the top concerns of mothers of infants and young children and they seek natural/nutritional solutions for a “happy tummy” (gut health).

Today, probiotics are largely used to reach some level of gut comfort in infants. However, there are constraints for that use in infants and young children.

First, this requires most of the time a dry format for the viability of the probiotic to deliver their metabolites in the gut and such a format is not the most appropriate for infant and young children.

Milk fermentation may lead to the production of various metabolites of potential interest for gut health but liquid milk fermentation with probiotics requires addition of starters/activators, leading to not viscous enough products with cheesy flavor notes that are often not preferred by infants and young children.

Abundance of proteins in diet leads to proteolytic fermentation in the colon promoting the production of branched short-chain fatty acids and ammonium. Those two byproducts of protein fermentation are not favorable for the host, while a high ratio saccharolytic/proteolytic fermentation is associated with better health outcomes including immune and gut health.

Thus, there is a need to find a nutritional solution in an acceptable product format which could beneficially contribute to Immune and Gut health of infants and young children consuming it. There is thus also an additional need for that nutritional solution to drive metabolism towards saccharolytic fermentation to promote immune and gut health.

The present inventors found a solution to the above mentioned technical problems by developing a process to prepare a product (nutritional composition) which comprises milk fermented with two probiotics (Bifidobacterium lactis—B. lactis and Lacticaseibacillus rhamnosus—L. rhamnosus) and that naturally delivers metabolites for gut benefits and immune health.

### SUMMARY OF THE INVENTION

The above mentioned problems are solved by a process to produce a fermented milk product, the fermented milk product prepared according to such process and a nutritional composition comprising the fermented milk product as set out in the attached claims.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 is a schematic representation of the process of making a fermented milk product according to the present invention.

FIG. 2 reports results of experiments of Example 4 and shows that fermentation with B. lactis AGAL NM97/09513 only increases the level of the free aminoacids serine, glycine, threonine, alanine and aspartate while fermentation with both B. lactis AGAL NM97/09513 and L. rhamnosus allows to decrease these levels.

FIG. 3 shows that milk fermentation by 2 probiotics according to the recent invention increases the levels of UMP (based on results in Example 5)

FIG. 4 shows that fermentation of milk allows to decrease the levels of some metabolites found associated with altered gut barrier function in EE (based on results of Example 5)

FIG. 5 shows results of Example 6. In particular, it shows that the nutritional composition according to the present invention induced an increase of Bifidobacteria.

FIG. 6 shows results of Example 6. In particular, it shows that all tested products significantly reduced production of ammonium.

FIG. 7 is a table summarizing the results obtained in the experiments of Example 6.

### DETAILED DESCRIPTION OF THE INVENTION

### Definitions

Within the context of the present invention, the term “fermented milk product” refers to dairy product that has been fermented with cultures of Bifidobacterium lactis and Lacticaseibacillus rhamnosus, for example B. Lactis AGAL NM97/09513 or B. lactis NCC2818 and L. rhamnosus AGAL NM97/09514 or L. rhamnosus LPR NCC4007.

L. rhamnosus AGAL NM97/09514 (HN001™) is commercially available and can be purchased for example under the name HOWARU® Protect EarlyLife (Rhamnosus) from Howaru (DuPont Danisco) catalogue name HN001™.

L. rhamnosus AGAL NM97/09514 was deposited at the Patent Culture Collection, Australian Government Analytical Laboratories (AGAL), 1 Suakin St, Pymble. NSW. 2073, Australia under deposit number NM/9709514. Within the context of the present invention, L. rhamnosus AGAL NM97/09514 is also referred to as L. rhamnosus HN001™ or L. rhamnosus HN001.

Lactobacillus rhamnosus NCC 4007 was deposited under the Budapest Treaty as Lactobacillus rhamnosus CGMCC 1.3724 in October 2004 with the China General Microbiological Culture Collection Center (CGMCC), Chinese Academy of Sciences, P.O. Box 2714, Beijing, China 100080.

B. lactis AGAL NM97/09513 (HN019™) is commercially available and can be purchased for example under the name HOWARU® Transit (Bifido) from Howaru (DuPont Danisco) catalogue name HN019™.

B. lactis AGAL NM97/09513 was deposited at the Patent Culture Collection, Australian Government Analytical Laboratories (AGAL), Australian Government Analytical Laboratories (AGAL), 1 Suakin St, Pymble. NSW. 2073, Australia as deposit number NM/9709514. Within the context of the present invention, B. lactis AGAL NM97/09513 is also referred to as B. lactis HN019™ or B. lactis HN019.

B. lactis NCC2818 was deposited under the Budapest treaty at the Collection Nationale de Cultures de Micro-Organisnes (CNCM, Institut Pasteur, 28 rue du Dr Roux, 75724 Paris Cedex 15, France): under the name Bifidobacterium lactis NCC 2818 access number CNCM I-3446 on Jun. 7, 2005.

Within the context of the present invention, the term “dairy product” means any product containing at least 15% milk (either in the form of whole, skimmed or partially skimmed milk).

Within the context of the present invention, the term “fermented milk powder” or “powdered fermented milk” refers to a fermented milk product as above defined which is in a powder format, for example a dairy product that has been fermented with cultures of B. Lactis and L. rhamnosus, for example of B. lactis AGAL NM97/09513 and L. rhamnosus AGAL NM97/09514 and then spray-dried to obtain a powder. The fermented milk powder according to the present invention may in some embodiments contain additiona ingredients such as for example pectin and cream powder.

Within the context of the present invention the term “sieving” refers to a well-known process in state of the art which is applied to the product in order to remove with a sieve the undesired particles with higher dimensions such as lumps larger than 2 mm, for example using a 2 mm sieve during such sieving step.

The expression “nutritional composition” means a composition which nourishes a subject. This nutritional composition is usually to be taken orally, and it usually includes a carbohydrate source, a lipid or fat source and a protein source. In one embodiment, the nutritional composition of the invention is a synthetic nutritional composition.

In a particular embodiment the composition of the present invention is a “synthetic nutritional composition”. The expression “synthetic nutritional composition” means a mixture obtained by chemical and/or biological means (i.e. the synthetic composition is not breast milk).

The word “fermentation” in the context of the present invention refers to the metabolic process that produces chemical changes in organic substrates through the action of bacterial enzymes substance. This metabolic process of fermentation leads to the production of metabolites.

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children. The term “fortifier” refers to liquid or solid nutritional compositions suitable for fortifying or mixing with human milk, infant formula, growing-up milk, or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The expression “weaning period” means the period during which the mother's milk is substituted by other food in the diet of an infant or young child.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or a growing-up milk or an infant cereal product or any other formulation designed for infant nutrition).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

### Process of Preparation of the Fermented Milk Product and Nutritional Compositions Comprising it

In one aspect, the present invention provides for a process for the preparation of a powdered fermented milk product which comprises:
a) heating skimmed milk concentrate to temperature above 65° C. for few seconds then to higher temperature then cooling to fermentation temperature;
b) fermenting the product of step a) with B. lactis AGAL NM97/09513 and L. rhamnosus AGAL NM97/09514 at a temperature ranging from 38 to 45° C., at a specific ratio of probiotics which ranges from: 99:1 to 1:99 and at pH ranging from 4.3 to 4.9;
c) cooling the product of step b) to a temperature below 10° C.;
d) heating product of step c) at a temperature ranging from 55 to 65° C., then immediately spray drying the product of step c) at a temperature ranging from 70 to 200° C.);
e) then optionally blending the product of step d) with cream powder and pectin and optionally sieving.

In one embodiment, first part of step a) is performed by heating skimmed milk concentrate to a temperature of at least 75° C. for few seconds, for example at least 15 seconds, then heating to higher temperature, for example at 85° C. In another embodiment, second part of step a) is performed by cooling to fermentation temperature ranging from 38 to 45° C., for example ranging from 41 to 43° C. In a further embodiment, step a) is performed by heating skimmed milk concentrate to a temperature of at least 75° C. for few seconds, for example at least 15 seconds, then heating to higher temperature, for example at 85° C., then cooling to fermentation temperature ranging from 38 to 45° C., for example ranging from 41 to 43° C.

In one embodiment, step b) is performed by fermenting the product of step a) with B. lactis AGAL NM97/09513 and L. rhamnosus AGAL NM97/09514 at a temperature ranging from 38 to 45° C., preferably ranging from 41 to 43° C.

In one embodiment, step b) is performed by fermenting the product of step a) with B. lactis AGAL NM97/09513 and L. rhamnosus AGAL NM97/09514 at a specific ratio of probiotics which ranges from 99:1 to 1:99. In one embodiment of the present invention, the ratio of probiotics B. lactis AGAL NM97/09513 and L. rhamnosus AGAL NM97/09514 is selected in the group consisting of: 99:1, 50:50; 75:25; 50:50; 25:75 1:99. In one further embodiment, the ratio of probiotics B. lactis AGAL NM97/09513 and L. rhamnosus AGAL NM97/09514 ranges from 65:53 to 85:15, for example 75:25.

In one embodiment, step b) is performed at a pH ranging from 4.3 to 4.9, for example ranging from 4.6 to 4.7.

In a further embodiment, step b) is performed by fermenting the product of step a) with B. lactis AGAL NM97/09513 and L. rhamnosus AGAL NM97/09514 at a temperature ranging from 38 to 45° C., preferably ranging from 41 to 43° C., using B. lactis AGAL NM97/09513 and L. rhamnosus AGAL NM97/09514 at a specific ratio which ranges from 65:53 to 85:15, for example 75:25, and step b) is performed at a pH ranging from 4.3 to 4.9, for example ranging from 4.6 to 4.7.

In one embodiment, Step c) is performed by cooling the product of step b) to a temperature below 10° C., to a temperature ranging from 4 to 10° C., for example from 4 to 8° C.

In one embodiment, step d) is performed by heating the product of step c) at a temperature ranging from 55 to 65° C., for example at 60° C., then immediately spray drying the product at a temperature ranging from 70 to 200° C.

In one embodiment, the temperature of inlet and outlet air of the spray dryer are ranging between 7° and 200° C.

In one embodiment, the fermented product is dried to a powder under step d) by spray drying using a centrifuge disk to disperse the fermented product of step c) to a controlled drop size spray.

In one embodiment of the present invention, a multi-stage-spray drier is used.

In another embodiment of the present invention, drying of the fermented milk product powder is finalized on an external vibrating fluidized bed.

In one embodiment of the present invention, it is provided a process for the preparation of a nutritional composition comprising a powdered fermented milk the process comprising preparing a powdered fermented milk according to the process of the present invention and comprising:
f) adding fruit and/or vegetable purees, one or more cereal flours and mixing until homogeneous;
g) adding fruit, for example lemon, juice to adjust pH to less than 4.2.
   h) optionally pasteurizing the mixture.

### Fermented Milk Product

In one aspect, the present invention provides for a fermented milk product which is obtainable by the process as above described.

In one embodiment of the present invention, the fermented milk product is a fermented milk powder.

In one embodiment of the present invention, the fermented milk product comprises skim milk powder, cream powder, B. lactis AGAL NM97/09513, L. rhamnosus AGAL NM97/09514, and pectin.

In one embodiment, the fermented milk powder according to the present invention contains additional ingredients such as for example pectin in an a mount ranging from 0 to 5% w/w, for example 1 to 3% w/w) and cream powder (in an amount ranging from 25 to 50% w/w, for example 30 to 40% w/w).

In one embodiment of the present invention, the fermented milk product comprises B. lactis AGAL NM97/09513 and L. rhamnosus AGAL NM97/09514 in a ratio ranging from 65:30 to 85:15, for example in a ratio of 75:25.

In one embodiment of the present invention, the fermented milk product is in a powder format.

### Nutritional Composition Comprising the Powdered Fermented Milk Product According to the Invention

In one embodiment of the present invention, a nutritional composition is provided which comprises the powdered fermented product according to the present invention. In one embodiment of the present invention, a nutritional composition is provided which comprises the powdered fermented product obtainable according to the process of the present invention.

In one embodiment of the present invention, the nutritional composition is a Baby food product, a follow up formula or growing up milk. In one embodiment, the nutritional composition of the present invention is a baby food product, a follow up formula or growing up milk which comprises a milk based portion.

In one embodiment of the present invention, nutritional composition is a synthetic nutritional composition.

In one embodiment of the present invention, the nutritional composition comprises a powdered fermented milk according to the present invention in an amount ranging from 1 to 5% w/w, fruit puree in an amount ranging from 80 to 99.7% w/w, vegetable puree in an amount ranging from 0 to 20% w/w, cereal flour in an amount ranging from 0.1 to 3% w/w, fruit juice concentrate in an amount ranging from 0.05 to 1% w/w, vegetable oil in an amount ranging from 0 to 3% w/w.

In one embodiment of the present invention, the nutritional composition comprises a fruit puree which is selected in the list consisting of: pear puree, banana puree, raspberry puree, strawberry puree, apple puree, prune puree, cherry puree, blueberry puree, blackberry puree, and mixtures thereof.

In one embodiment of the present invention, the nutritional composition comprises a vegetable puree which is selected in the list consisting of: spinach puree, sweet potato puree, and mixtures thereof.

In one embodiment of the present invention, the nutritional composition comprises a cereal flour which is selected in the list consisting of: oat flour, quinoa flour, barley flour, and mixtures thereof.

In one embodiment of the present invention, the nutritional composition comprises a fruit juice concentrate which is lemon juice concentrate.

In one embodiment of the present invention, the nutritional composition comprises a vegetable oil which is walnut oil.

### Uses and Advantages of the Fermented Milk Product According to and Nutritional Composition Comprising it

The inventors have surprisingly observed a number of unexpected advantages and benefits for the fermented milk product according to the invention, for example the powdered fermented milk product according to the present invention, and for the nutritional compositions comprising it.

### Palatability of Powdered Fermented Product and Nutritional Compositions Comprising it

As above mentioned, liquid milk fermentation with probiotics requires the addition of starters/activators, leading to not viscous enough products with cheesy flavor notes.

The results shown in Example 3 indicate that a nutritional composition comprising a powdered fermented milk powder according to the present invention shows optimal palatability. The effect is particularly prominent when compared with palatability of a corresponding nutritional composition based on liquid fermented milk product with a single microorganism (B. lactis).

In one embodiment, the present invention provides for the use of a powdered fermented milk product according to the present invention for improved palatability. In one embodiment of the present invention, it is also provided the use of a powdered fermented milk product according to the present invention for improved palatability of a nutritional composition comprising it.

Optimal Amino-Acid Absorption from Powdered Fermented Product and Nutritional Compositions Comprising it

Fermentation usually leads to protein breakage and liberation of peptides, tri-, di-peptides and single amino acids. Yet, amino-acids do not represent the form of nitrogen which is easiest to absorb.

The inventors surprisingly found that the fermented milk powder which was fermented with the 2 probiotics according to the present invention, for example at the ratio 75:25, led to the decrease in several amino acid compared to a fermented milk product with a single probiotic. Results are reported in Example 4.

In one embodiment, the present invention thus provides for the use of a powdered fermented milk product according to the present invention for promoting nutrients' absorption in the gut, for example amino acids absorption. In one embodiment of the present invention, it is also provided the use of nutritional composition comprising a powdered fermented milk product according to the present invention for promoting nutrients' absorption in the gut, for example amino acids absorption.

In one embodiment, a powdered fermented milk product according to the present invention is provided for use in promoting nutrients' absorption in the gut, for example amino acids absorption. In one embodiment of the present invention, it is also provided a nutritional composition comprising a powdered fermented milk product according to the present invention for use in promoting nutrients' absorption in the gut, for example amino acids absorption.

In one embodiment, the present invention thus provides for the use of a powdered fermented milk product according to the present invention in the manufacture of a nutritional composition for promoting nutrients' absorption in the gut, for example amino acids absorption.

In one embodiment, the present invention thus provides for a method for promoting nutrients' absorption in the gut, for example amino acids absorption in a subject in need thereof, the method comprising administering to the subject a powdered fermented milk product according to the present invention or a nutritional composition comprising it.

In one embodiment of the present invention, the use of the powdered fermented milk powder and nutritional composition comprising it for promoting nutrients' absorption is a non-therapeutic use.

### Benefits on Gut Health for the Powdered Fermented Product and Nutritional Compositions Comprising it

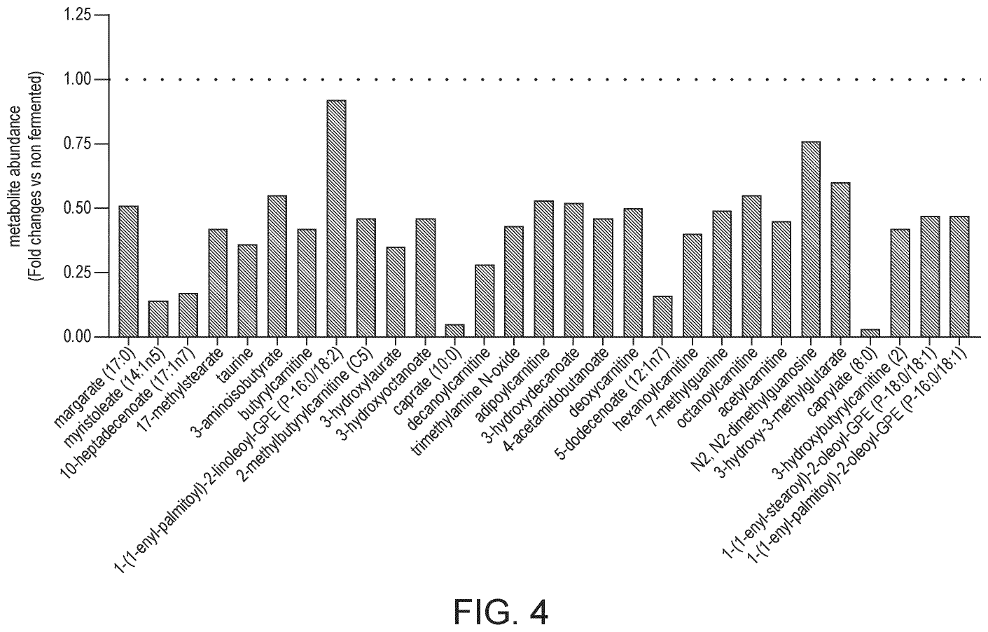

The inventors surprisingly found that in the fermented milk powder which was fermented with the 2 probiotics according to the present invention, it was observed:
An increase of Uridine-5′ MonoPhosphate (UMP) which is a component involved in gut and immune health, especially in preventing weaning-associated diarrhea in a preclinical model. UMP is naturally present in milk, including human milk, and is known to promote intestinal maturation and repair (FIG. 3) as shown in Li et al. Food Funct., 2019.
that fermented milk according to the present invention present decreased levels of certain metabolites (e.g. 1-(1-enyl-palmitoyl)-2-oleoyl-GPE (P-16:0/18:1), 3-hydroxybutyrylcarnitine, and caprylate, among others) that have been described by Semba et al. EBioMedicine 17 (2017) to be associated with altered gut barrier function in the serum of subjects suffering Environmental Enteropathy (FIG. 4)

Results are reported in Example 5.

Overall, the results show that the fermented milk powder according to the present invention contains metabolites which are beneficial for the gut health of the host consuming the fermented milk powder or a nutritional composition comprising it.

In one embodiment, the present invention thus provides for the use of a powdered fermented milk product according to the present invention for promoting gut health, for example for preventing diarrhea. In one embodiment of the present invention, it is also provided the use of nutritional composition comprising a powdered fermented milk product according to the present invention for promoting gut health, for example for preventing diarrhea.

In one embodiment, a powdered fermented milk product according to the present invention is provided for promoting gut health, for example for preventing diarrhea. In one embodiment of the present invention, it is also provided a nutritional composition comprising a powdered fermented milk product according to the present invention for promoting gut health, for example for preventing diarrhea.

In one embodiment, the present invention thus provides for the use of a powdered fermented milk product according to the present invention in the manufacture of a nutritional composition for promoting gut health, for example for preventing diarrhea.

In one embodiment, the present invention thus provides for a method for p for promoting gut health, for example for preventing diarrhea in a subject in need thereof, the method comprising administering to the subject a powdered fermented milk product according to the present invention or a nutritional composition comprising it.

In one embodiment of the present invention, the use of the powdered fermented milk powder and nutritional composition comprising it for promoting gut health, for example for preventing diarrhea is a non-therapeutic use.

### Promotion of Beneficial Gut Microbiota and Immune Health for the Powdered Fermented Product and Nutritional Compositions Comprising it

When food is ingested, most of the proteins are usually absorbed in the upper GI tract and do not reach the colon. On the contrary, fibers are not digested and reach the colon in an intact form, where they will be fermented. This is the optimal situation for health. Yet, in our recent decade, the decrease in fiber in the diet and increase in protein consumption can lead some proteins to be fermented in the colon.

There are two colonic microbial fermentation types:

1) The saccharolytic fermentation of carbohydrate or fibers: this leads to formation of large amount of carbohydrate metabolites like short-chain fatty acids (SCFA), mainly acetate, propionate, and butyrate. SCFA benefits are local in the gut layer and contribute to gut health. SCFA decrease fecal pH and thus facilitate luminal mineral absorption.

SCFA can act directly on immune cells, such as dendritic cells, T cells, B cells and have been shown to increase immune protection against both viral and bacterial infection in the gut or in the lungs. SCFA are also associated to numerous other remote benefits for metabolic, brain, and skin health.

Bifidobacteria can grow on specific carbohydrate and have been shown to directly contribute to immune protection.

2) The proteolytic fermentation of protein or peptides or aminoacids.

Proteolytic fermentation also leads not only to the production of branched-chain fatty acids but also to potentially toxic metabolites such as phenolic compounds, sulfur-containing compounds, amines, and ammonium. Ammonium allow the growth of bacterial pathogens and high amount of ammonia are associated with increased chance of bacterial enterocolitis or other type of bacterial and viral infections.

As such, an increased saccharolytic to proteolytic fermentation ratio in the colon is thus considered favorable for immune health.

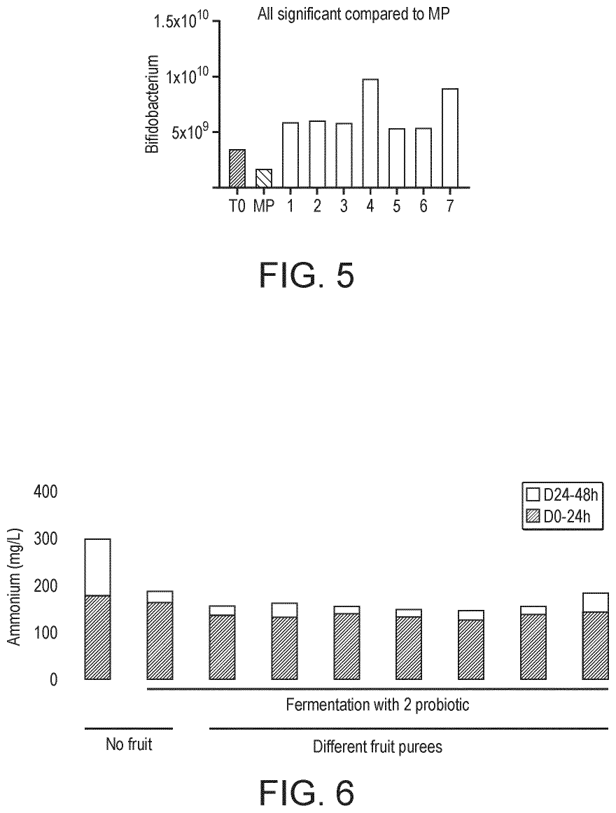

The inventors surprisingly found that the nutritional composition according to the present invention induced an increase of Bifidobacteria compared to the fermented milk powder alone. Significantly higher Bifidobacterium spp. levels were observed with the nutritional composition compared with PFM (fermented milk powder) after 24 h and/or 48 h (FIG. 5).

Interestingly, ammonium production was highly reduced by the fermented milk, and this was conserved with the addition of the fruit puree suggesting that milk fermentation decreases proteolytic fermentation, and ammonium and branched-chain SCFA production associated with increased risk for infection.

Results are reported in Example 6.

In one embodiment, the present invention thus provides for the use of a powdered fermented milk product according to the present invention for promoting gut beneficial microbiota and immune health. In one embodiment of the present invention, it is also provided the use of nutritional composition comprising a powdered fermented milk product according to the present invention for promoting gut beneficial microbiota and immune health.

In one embodiment, a powdered fermented milk product according to the present invention is provided for for promoting gut beneficial microbiota and immune health. In one embodiment of the present invention, it is also provided a nutritional composition comprising a powdered fermented milk product according to the present invention for promoting gut beneficial microbiota and immune health.

In one embodiment, the present invention thus provides for the use of a powdered fermented milk product according to the present invention in the manufacture of a nutritional composition for promoting gut beneficial microbiota and immune health.

In one embodiment, the present invention thus provides for a method for for promoting gut beneficial microbiota and immune health in a subject in need thereof, the method comprising administering to the subject a powdered fermented milk product according to the present invention or a nutritional composition comprising it.

In one embodiment of the present invention, the use of the powdered fermented milk powder and nutritional composition comprising it for promoting gut beneficial microbiota and immune health is a non-therapeutic use.

### EXPERIMENTAL SECTION

### Example 1

### Preparation of a Fermented Milk Product (Powder)

A fermented milk powder according to the recipe presented in Table 1 was prepared as follows.

Skimmed milk concentrate was heated at 75° C. for 15 sec, then at 85° C. for 1 sec, then cooled to fermentation temperature in the range of 42±1° C.; B. lactis AGAL NM97/09513 and L. rhamnosus AGAL NM97/09514 were added at a 75:25 ratio and fermented until the pH of 4.6±0.3 was reached and then cooled to 8° C. The product was then heated at a temperature of 60±5° C. then immediately spray dried. The Powder Fermented Milk was then blended with cream powder and pectin, then sieving with a 2 mm filter.

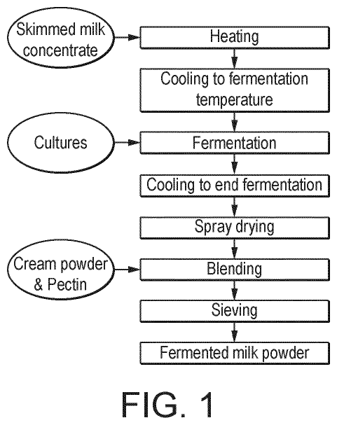

The process of making it is also schematically represented in FIG. 1.

TABLE 1
Fermented milk recipe

| Ingredients | % |
|---|---|
| Concentrated Skim milk | 63 |
| Cream powder | 36 |
| Bifidobacterium lactis | 325 DCU/100 L |
| AGAL NM97/09513 | |
| Lactobacillus rhamnosus | 83 DCU/100 L |
| AGAL NM97/09514 | |
| Pectin | 0.15 |
| Total | 100.00 |

### Example 2

### Preparation of Nutritional Composition Comprising a Fermented Milk

Nutritional compositions (Baby Food products in the form of purees) were prepared comprising the fermented milk powder prepared as described in Example 1. Nutritional compositions were prepared according to several recipes whose ingredients are reported in Tables 2 to 8 below. Table 9 also describes ranges for such ingredients expressed as percentages in weight in a generic recipe.

The Powder Fermented Milk prepared as described in Example 1 was blended with cream powder and pectin, then sieving with a 2 mm filter before adding purees in the tank, and cereal flours and mixing until homogeneous. Fruit (lemon) juice was added to adjust pH then the product was transferred to a holding tank for pasteurization hot filling. at high temperature, followed by nitrogen injection, X-ray and packaging into a carton box.

TABLE 2
(Recipe n. 1)
Ingredient

Powder Fermented Milk
Organic Pear Puree
Organic Apple Puree
Organic Banana Puree
Organic Raspberry Puree
Organic Strawberry Puree
Oat flour
Lemon Juice Concentrated

TABLE 3
(Recipe n. 2)

Powder Fermented Milk
Organic Apple Puree
Organic Prune Puree
Organic Banana Puree
Oat Flour
Quinoa Flour
Lemon Juice Concentrated

TABLE 4
(Recipe n. 3)

Powder Fermented Milk
Organic Cherry Puree
Organic Banana Puree
Organic Apple Puree
Organic Strawberry Puree
Oat Flour
Lemon Juice Concentrated

TABLE 5
(Recipe n. 4)

Powder Fermented Milk
Organic Apple Puree
Organic Banana Puree
Organic Blueberry Puree
Organic Blackberry Puree
Oat flour
Quinoa Flour
Lemon Juice Concentrated

TABLE 6
(Recipe n. 5)

Powder Fermented Milk
Organic Pear Puree
Organic Apple Puree
Spinach Puree
Oat Flour
Quinoa Flour
Lemon Juice Concentrated

TABLE 7
(Recipe n. 6)

Powder Fermented Milk
Organic Apple Puree
Organic Sweet Potato Puree
Peach Puree
Walnut oil
Barley Flour
Lemon Juice Concentrated

TABLE 8
(Recipe n. 7)

Powder Fermented Milk
Organic Banana Puree
Organic Raspberry Puree
Organic Pear Puree
Walnut oil
Barley Flour
Lemon Juice Concentrated

TABLE 9
(generic Recipe)

| | |
|---|---|
| Powder Fermented Milk | 1-5% |
| Fruit Puree | 80-99.7% |
| Vegetable puree | 0-20% |
| Cereal flour | 0.1-3% |
| Fruit juice concentrate | 0.05-1% |
| Vegetable oil | 0-3% |
| Total | 100.00 |

### Example 3

Probiotic Selection and their Ratio to Achieve Optimal Palatability in Fermented Milk Powder and Nutritional Compositions Comprising it

A milk product was fermented according to the process above described first with different ratio of probiotics B. lactis:L. rhamnosus (100:0, 75:25, 50:50, 25:75, 0:100)

Results showed that the best palatability and texture were obtained at ratio of probiotic of 75:25.

The results below in Table 10 show result in the final product recipes for the ratio B. lactis:L. rhamnosus 25:75 and 100:0, indicating that the ratio 75:25 provide a preferable flavor

TABLE 10

| | Purees with fermented | Purees with fermented |
|---|---|---|
| | milk (2 cultures 75:25) | milk (1 culture B. lactis) |
| Recipe | Taste/Flavor | Taste/Flavor |
| Pear-Fermented | Balanced | Sour |
| Milk-Banana- | | |
| Raspberry- | | |
| Strawberry and | | |
| Oat | | |
| Apple | excellent | Sour, less fruity, |
| Prune Fermented | fruity flavor | fermented fruit |
| Milk-Banana with | | |
| Oat and Quinoa | | |
| Banana- | Strong cherry, | Sour |
| Fermented Milk- | fresh fruit | |
| Cherry- | | |
| Strawberry with | | |
| Oat | | |
| Apple- | Preferred | Sour |
| Fermented Milk- | | |
| Banana- | | |
| Blackberry- | | |
| Blueberry with | | |
| Oat and Quinoa | | |
| Pear-Fermented | / | Strong bitter, |
| Milk-Apple- | | sour |
| Spinach with Oat | | |
| and Quinoa | | |
| Apple-Fermented | raw sweet | Roast sweet |
| Milk-Sweet | potato | potato |
| Potato-Peach | | |
| with Walnut and | | |
| Barley | | |
| Banana- | Balanced | / |
| Fermented Milk- | | |
| Pear | | |
| Raspberry with | | |
| Walnut and | | |
| Barley | | |

### Example 4

Optimal Amino Acid Absorption in Fermented Milk Powder and/or Nutritional Compositions Comprising it

The presence of amino acid before and after fermentation was investigated for the fermented milk powder according to the invention. Fermentation usually leads to protein breakage and liberation of peptides, tri-, di-peptides and free amino acids. Yet, amino-acids do not represent the form of nitrogen which is easiest to absorb. All samples were maintained at −80° C. until processed.

Samples were prepared using the automated MicroLab STAR® system from Hamilton Company and protein were precipitated with methanol. The resulting extract was divided into five fractions: two for analysis by two separate reverse phases (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent.

Several types of controls were analysed in concert with the experimental samples: a pooled matrix sample generated by taking a small volume of each experimental sample (or alternatively, use of a pool of well-characterized human plasma) served as a technical replicate throughout the data set; extracted water samples served as process blanks.

Surprisingly, the fermented milk powder fermented with the 2 probiotics at the ratio 75:25 led to the decrease in several amino acid compared to fermentation milk with the single probiotic indicating that less free amino acid were released from the fermentation with the 2 probiotics. The results suggest that the metabolization of nitrogen substrate was changed by the addition of the L. rhamnosus and suggests a better nitrogen availability in the PFM generated with a probiotic ration of 75:25

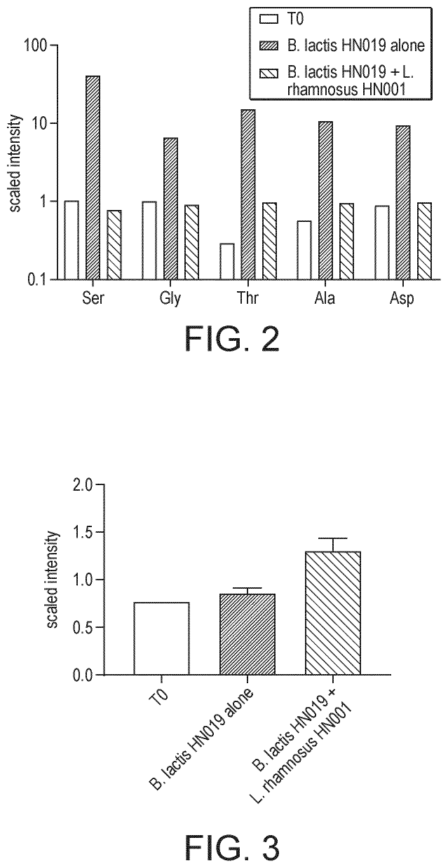

FIG. 2 shows that fermentation with B. lactis AGAL NM97/09513 only increases the level of the free aminoacids serine, glycine, threonine, alanine and aspartate. Fermentation with both B. lactis AGAL NM97/09513 and L. rhamnosus allows to decrease these levels.

### Example 5

### Nutritional Compositions According to the Present Invention are Beneficial for Diarrhea Prevention and Gut Health

The metabolites before and after fermentation were investigated for the fermented milk powder according to the invention and for a milk product fermented only with one probiotic (B. lactis).

Samples were inventoried and immediately stored at −80° C. All samples were maintained at −80° C. until processed.

Samples were prepared using the automated MicroLab STAR® system from Hamilton Company and protein were precipitated with methanol. The resulting extract was divided into five fractions: two for analysis by two separate reverse phases (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent.

Several types of controls were analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks.

Under these conditions, it was observed:
1) An increase of Uridine-5′ MonoPhosphate (UMP) which is a component involved in gut and immune health, especially in preventing weaning-associated diarrhea in a preclinical model. UMP is naturally present in milk, including human milk, and is known to promote (Li et al. Food Funct., 2019, 10, 4081) intestinal maturation and repair (FIG. 3).
2) that fermented milk according to the present invention present decreased levels of metabolites e.g. 1-(1-enyl-palmitoyl)-2-oleoyl-GPE (P-16:0/18:1), 3-hydroxybutyrylcarnitine, and caprylate, among others) that have been found associated with altered gut barrier function (Semba et al. EBioMedicine 17 (2017) 57-66) in the serum of subjects suffering Environmental Enteropathy (FIG. 4)

Overall, these results show that the fermented milk powder according to the present invention contains metabolites which are beneficial for the gut health of the host consuming the fermented milk powder or a nutritional composition comprising it.

### Example 6

### Nutritional Compositions Comprising the Fermented Milk Powder According to the Present Invention are Beneficial for Gut Microbiota and Immune Health

The fermented powder milk and the final recipe were tested in a model of in vitro short term colonic fermentation, and changes in pH, ammonium, SCFA as well as microbiota composition were monitored.

Prior to the colonic incubations, all products were subjected to in vitro digestion consisting of a full passage through the oral, gastric, and small intestinal phase, the latter involving absorption (Dupont D. Crit Rev Food Sci Nutr 2019). This was considered important as these products contain a fraction of digestible compounds that, in vivo, is absorbed at the level of the small intestine following the conversion to small molecules. Simulation of small intestinal absorption via dialysis enables the removal of small molecules from intestinal digesta. To do so, membranes with 3.5 kDa cutoff were used.

The short-term colonic incubation screening assay consisted of a colonic incubation of a single dose of a (predigested) test product under conditions representative for the colon region of an 8-12-month-old infant donor.

Product dosage was normalized for moisture content, so that a concentration of 5 g dry matter (DM)/L was obtained in the colonic reactors at the start of the incubation. However, as a dialysis step was performed prior to colonic fermentation, actual product concentrations in the reactors were lower than 5 g DM/L, depending on the degree of digestion and absorption of low molecular weight compounds during upper GI simulation.

An assessment of the changes in pH, gas, SCFA, ammonium and lactate production was made. Targeted qPCR was performed to assess the impact of treatments on the gut microbial community at 0 h, 6 h, 24 h and 48 h.

Changes in community composition was assessed by targeted qPCR.

The analysis was performed at 0 h, 24 h and 48 h to assess treatment effects on Firmicutes, Bacteroidetes, Bifidobacterium spp., Lactobacillus spp. and Akkermansia muciniphila.

All tested recipes (recipes 1 to 7 as above described in Example 2) induced an increase of Bifidobacteria compared to the fermented milk powder alone. Significantly higher Bifidobacterium spp. Levels were observed with nutritional compositions comprising the fermented milk powder compared to PFM (fermented milk powder) after 24 h and/or 48 h (FIG. 5). The strongest stimulatory effects were observed for 2 products. Enrichment of Bifidobacteria was mostly observed between 0-24 h.

As far as Lactobacillus increase is concern, the fermented milk allows better survival or growth of colonic lactobacillus compared to the non-fermented milk.

All products, except TO significantly reduced production of ammonium (FIG. 6). T0 was associated with increases in ammonium levels suggesting that the fermentated milk and the fermentation product all displayed an improved saccharolytic:proteolitic ratio.

Ammonium production was highly reduced by the fermented milk, and this was conserved with the addition of the fruit puree suggesting that the fermentation of the milk decreased the proteolytic fermentation and ammonium production (and branched-chain SCFA), associated with increased risk of infection.

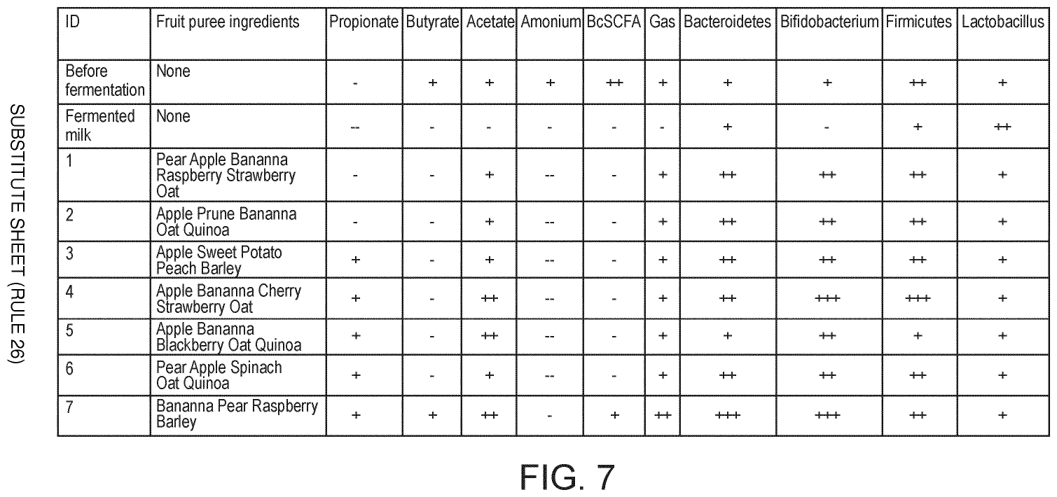

The results of the present experiment are summarized in FIG. 7.

Bifidobacteria and Lactobacilli are regarded as beneficial saccharolytic bacteria, capable of producing high concentrations of lactate and acetate (Rios-Covián D. Applied and Environmental Microbiology 79(23) 2013). Lactate is an important metabolite because of its antimicrobial properties, but also (together with acetate) because it is the driver of a series of trophic interactions with other bacteria, resulting in the production of downstream metabolites (A. N. Vlasova; Vet Immunol Immunopathol. 2016).

Less abundant SCFA include branched SCFA (isobutyrate, isovalerate and isocaproate). Ammonium and branched SCFA production results from proteolytic microbial activity, which is associated with formation of toxic by-products such as p-cresol. Therefore, high branched SCFA and ammonium production in the colon has been associated with detrimental health effects. As a consequence, products that reduce branched SCFA and ammonium production are considered health-beneficial specifically for gut health and immune protection.

### Other Embodiments of the Invention

i) A process for the preparation of a powdered fermented milk product which comprises:
   a) heating skimmed milk concentrate to temperature above 65° C. for few seconds then to higher temperature then cooling to fermentation temperature;
   b) fermenting the product of step a) with B. lactis and L. rhamnosus at a temperature ranging from 38 to 45° C., at a specific ratio of probiotics which ranges from: 99:1 and 1:99 at pH ranging from 4.3 to 4.9;
   c) cooling the product of step b) to a temperature below 10° C.;
   d) heating product of step c) at a temperature ranging from 55 to 65° C., then immediately spray drying the product of step c) at a temperature ranging from 70 to 200° C.);
   e) then optionally blending the product of step d) with cream powder and pectin, and optionally sieving.
ii). Powdered fermented milk product obtainable by the process described in embodiment i).
iii). Powdered fermented milk product obtainable by the process of claim 1 which comprises concentrated skim milk, cream powder, B. lactis AGAL NM97/09513, L. rhamnosus AGAL NM97/09514 and pectin.
iv). Process for the preparation of a nutritional composition comprising a powdered fermented milk product according to any of embodiments ii) or iii) which comprises:
   a) heating skimmed milk concentrate to temperature above 65° C. for few seconds then to higher temperature then cooling to fermentation temperature;
   b) fermenting the product of step a) with B. lactis and L. rhamnosus at a temperature ranging from 38 to 45° C., at a specific ratio of probiotics which ranges from: 99:1 and 1:99 at pH ranging from 4.3 to 4.9;
   c) cooling the product of step b) to a temperature below 10° C.;
   d) heating product of step c) at a temperature ranging from 55 to 65 deg C., then immediately spray drying the product of step c) at a temperature ranging from 70 to 200° C.);
   e) then optionally blending the product of step d) with cream powder and pectin, and optionally sieving;
   f) adding fruit and/or vegetable purees, one or more cereal flours and mixing until homogeneous;
   g) adding fruit (lemon) juice to adjust pH to less than 4.2;
   h) optionally pasteurizing.
v). Use of a fermented milk product according to embodiment ii) for promoting gut health, immune health and/or beneficial microbiota in the gut.
vi). A nutritional composition which comprises a fermented milk product obtainable according to the process described in embodiment i).
vii). nutritional composition according to embodiment iv) which comprises (recipes)
viii). Use of a nutritional composition according to claims vii) or viii) for promoting gut Health, Immune health and/or beneficial microbiota in the gut.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. A process for the preparation of a powdered fermented milk product which comprises:
a) heating skimmed milk concentrate to temperature above 65° C. for few seconds then to higher temperature then cooling to fermentation temperature;
b) fermenting the product of step a) with B. lactis and L. rhamnosus at a temperature ranging from 38 to 45° C., at a specific ratio of probiotics which ranges from: 99:1 and 1:99 at pH ranging from 4.3 to 4.9;
c) cooling the product of step b) to a temperature below 10° C.; and
d) heating product of step c) at a temperature ranging from 55 to 65° C., then immediately spray drying the product of step c) at a temperature ranging from 70 to 200° C.

2. A process according to claim 1 wherein B. lactis and L. rhamnosus are respectively the strains B. lactis AGAL NM97/09513 and L. rhamnosus AGAL NM97/09514.

3. A process according to claim 1 wherein the fermentation step b) is carried out with a probiotic mixture consisting of B. lactis and L. rhamnosus.

4. A process according to claim 1 which is for the preparation of a nutritional composition comprising a powdered fermented milk product.

5-6. (canceled)

7. A nutritional composition which comprises a fermented milk product obtainable according to the process comprising:
a) heating skimmed milk concentrate to temperature above 65° C. for few seconds then to higher temperature then cooling to fermentation temperature;
b) fermenting the product of step a) with B. lactis and L. rhamnosus at a temperature ranging from 38 to 45° C., at a specific ratio of probiotics which ranges from: 99:1 and 1:99 at pH ranging from 4.3 to 4.9;
c) cooling the product of step b) to a temperature below 10° C.; and
d) heating product of step c) at a temperature ranging from 55 to 65° C., then immediately spray drying the product of step c) at a temperature ranging from 70 to 200° C.

8. Nutritional composition according to claim 7 which comprises powdered fermented milk in an amount ranging from 1 to 5% w/w of the composition, fruit puree in an amount ranging from 80 to 99.7% w/w of the composition, vegetable puree in an amount ranging from 0 to 20% w/w of the composition, cereal flour in an amount ranging from 0.1 to 3% w/w of the composition, fruit juice concentrate in an amount ranging from 0.05 to 1% w/w of the composition and vegetable oil in an amount ranging from 0 to 3% w/w of the composition.

9-12. (canceled)

13. A method for promoting gut Health, Immune health and/or beneficial microbiota in the gut in a subject in need thereof, the method comprising administering to the subject a powdered fermented milk product obtainable according to the process comprising:
a) heating skimmed milk concentrate to temperature above 65° C. for few seconds then to higher temperature then cooling to fermentation temperature;
b) fermenting the product of step a) with B. lactis and L. rhamnosus at a temperature ranging from 38 to 45° C., at a specific ratio of probiotics which ranges from: 99:1 and 1:99 at pH ranging from 4.3 to 4.9;
c) cooling the product of step b) to a temperature below 10° C.; and
d) heating product of step c) at a temperature ranging from 55 to 65° C., then immediately spray drying the product of step c) at a temperature ranging from 70 to 200° C.
